(19) United States
(12) Patent Application Publication
(10) Pub. No.: US 2010/0055181 A1
(43) Pub. Date: Mar. 4, 2010

(54) CONTROLLED RELEASE DOSAGE FORMS OF ZOLPIDEM

(75) Inventors: Nilesh Bhandari, Pune (IN); Vineeth Raghavan, Pune (IN); Surya Kumar Jayanthi, Pune (IN); Himadri Sen, Pune (IN)

Correspondence Address: VOLPE AND KOENIG, P.C., UNITED PLAZA, SUITE 1600, 30 SOUTH 17TH STREET, PHILADELPHIA, PA, 19103, US

(73) Assignee: LUPIN LIMITED, Mumbai, MM (IN)

(21) Appl. No.: 12/517,487

(22) PCT Filed: Feb. 7, 2007
(86) PCT No.: PCT/IN2007/000046

§ 371 Date: Jun. 3, 2009

(30) Foreign Application Priority Data
Dec. 18, 2006 (IN) 1368/KOL/2006

(51) Int. Cl. A61K 9/32; A61K 31/437; A61K 9/20; A61K 9/10; A61K 9/16

(52) U.S. Cl. 424/482; 514/300; 424/464; 424/484; 424/490

(57) ABSTRACT

A controlled release dosage forms comprising zolpidem or a salt thereof to release zolpidem to induce rapid onset of sleep, and continue to release zolpidem in a controlled manner to maintain effective plasma concentrations over an extended period of time to improve sleep maintenance. The pharmaceutical controlled-release dosage form of zolpidem or a salt thereof having a dissolution profile when measured in a type I or II dissolution apparatus according to the U.S. Pharmacopoeia in 0.01M hydrochloric acid buffer at 37° C., such that less than 40% is released at the end of 30 minutes.

### FIELD OF THE INVENTION

The present invention relates to novel controlled release dosage forms comprising zolpidem or a salt thereof to release zolpidem to induce rapid onset of sleep, and continue to release zolpidem in a controlled manner to maintain effective plasma concentrations over an extended period of time to improve sleep maintenance.

### BACKGROUND OF THE INVENTION

Insomnia is a condition related to abnormalities within diurnal rhythms. Early treatments for insomnia commonly employed central nervous system (CNS) depressants such as barbiturates. These compounds typically have long half-lives and have a well-known spectrum of side effects, including lethargy, confusion, depression and next day hangover effects. In addition, chronic use has been associated with a high potential for addiction involving both physical and psychological dependence. Benzodiazepine classes of sedative-hypnotic agents were employed for the treatment of insomnia. This class of compounds produces a calming effect that results in a sleep-like state in humans and animals, with a greater safety margin than prior hypnotics. However, many benzodiazepines possess side effects that limit their usefulness in certain patient populations. These problems include synergy with other CNS depressants (especially alcohol), the development of tolerance upon repeat dosing, rebound insomnia following discontinuation of dosing, hangover effects the next day and impairment of psychomotor performance and memory.

More recent treatments for insomnia have used non-benzodiazepine compounds. These compounds include Zolpidem, Zileuteon and zalepelon. Zolpidem is a suitable short acting hypnotic for the controlled-release dosage form according to the present invention. Zolpidem is a hypnotic from the therapeutical class of imidazopyridines. It is administrated orally by means of a tablet or other solid dosage form. Zolpidem was first disclosed in U.S. Pat. No. 4,382,938. It is marketed in US under the brand name Ambien® tablets and Ambien® CR tablets.

Immediate release dosage forms of zolpidem provide a burst of drug substance shortly after ingestion, to induce rapid onset of sleep. Whereas such dosage forms address the latency to sleep problem, unless the drug substance has a long half life, in order to maintain effective blood plasma concentration levels over an extended period of time, patients experiencing short sleep duration or frequent nocturnal awakening events will need to take further dosage forms during the night to maintain sleep.

Modified release dosage forms produce an initial burst of drug substance to induce rapid onset of sleep, and continue to release drug substance in a controlled manner to maintain effective plasma concentrations over an extended period of time to improve sleep maintenance. A potential disadvantage of this approach is the time to clearance of the active substance from a patient's system. Drug substance still present at effective levels can cause hangover effects upon wakening.

A particular modified release dosage form is described in U.S. Pat. No. 6,485,746. In this patent there is described a formulation of a sedative-hypnotic compound that provides a pulsatile release profile in vivo whereby upon administration the drug substance is released rapidly to provide a maximum plasma concentration within 0.1 to 2 hours following administration. Thereafter, plasma concentration passes through a minimum at about 2 to 4 hours post administration, before a second pulse delivers a second maximum plasma concentration at about 3 to 5 hours. Finally, after 8 hours there remains a plasma concentration that represents no more than 20% of the plasma concentration of the second maximum.

The above invention provides fluctuations in plasma concentration of drug. U.S. Pat. No. 6,541,531 discloses an oral pharmaceutical controlled-release dosage form adapted to releases the major portion (40-70%) of the total drug in immediate release phase for a maximum duration of 30 minutes and the remaining 30% of the drug remains for controlled release in between 2 and 6 hours. The invention provides a wide range of plasma concentration of drug in IR phase and small portion for controlled release, which is not sufficient to eliminate night awakening episodes.

The above patent has a biphasic profile wherein the majority of drug is released in the initial 30 minutes and very less amount is available for the controlled release over an extended period of time.

U.S. Pat. No. 6,638,535 relates to a modified release pellet composition of Zolpidem wherein the pellets are made of microcrystalline cellulose and has a characteristic release profile.

This patent has a disadvantage as it is expensive, time consuming and difficult to reproduce.

Since the biological process associated with the sleep (homeostatic process) is believed to be a primary driving force in creating in patients the need for sleep. For an individual having a bedtime of around 11 p.m., this drive weakens in the early morning hours, e.g. around 3 a.m., and is further exacerbated by a circadian alert pulse around 5 a.m. that is believed to be an additional driver to wakefulness for patients. Since the average sleep time for an individual in around 6 hours a pharmaceutical composition that maintains controlled release profile extending beyond 6 hours is required.

Thus the current invention describes a pharmaceutical dosage form that provides rapid onset of sleep and extended sleep duration for a time period of 2.5-7.5 hours and more importantly reduces or eliminates nocturnal awakening events.

The present invention proposes dosage forms of zolpidem or a salt thereof whose complete dissolution time, defined as the time for release of 90% of the total amount of drug is between 2.5 and 7.5 hours and preferably between 3.0 to 4.5 hours.

### BRIEF DESCRIPTION OF THE INVENTION

Zolpidem is a suitable short acting hypnotic for the controlled-release dosage form according to the present invention. Zolpidem is a hypnotic from the therapeutical class of imidazopyridines. It is administrated orally by means of a tablet or other solid dosage form. Zolpidem acts rapidly. Indeed pharmacokinetic and pharmacodynamic data show that zolpidem has both a rapid absorption and onset of hypnotic action. Its bioavailability is 70% following oral administration and demonstrates linear kinetics in the therapeutical dose range, which lies between 5 and 10 mg in conventional forms, peak plasma concentration is reached at between 0.5 and 3 hours, the elimination half-life is short, with a mean of 2.4 hours and a duration of action of up to 6 hours. For reasons of simplicity, in the absence of contrary indication, within the whole description “zolpidem” or the “drug” means zolpidem per se as well as its salts. The preferred salt of zolpidem is zolpidem hemitartrate.

Therefore, as a first object, the present invention provides controlled-release dosage forms comprising zolpidem or salts thereof adapted to release over a predetermined time period.

A suitable dissolution test is where the measurement is carried out in a type II dissolution (50 rpm) apparatus or type I dissolution (100 rpm) apparatus according to U.S. pharmacopoeia in aqueous buffer at 37.degree. C., or variations on this as well known to one who is skilled in the art.

The present invention proposes controlled release dosage forms of zolpidem or a salt thereof wherein the complete dissolution time that is the time for release of 90% of the total amount of the drug is between 2.5-7.5 hours, preferably between 3.0-4.5 hours.

Yet another object of the invention proposes controlled release dosage forms of zolpidem or a salt thereof wherein less than 40% is released at the end of 30 minutes.

Yet another object of the invention proposes controlled release dosage forms of zolpidem or a salt thereof, which exhibits a mean AUC₀₋ₜ in the range from 255 ng·hr/ml to 1610 ng·hr/ml in fasted conditions.

Yet another object of the invention proposes controlled release dosage forms of zolpidem or a salt thereof, which exhibits a mean Cₘₐₓ in the range from 65 ng/ml to 230 ng/ml in fasted conditions.

### BRIEF DESCRIPTION OF THE DRAWINGS

FIG. 1(a) shows a release profile of controlled release dosage forms of zolpidem of example 1, in Type I USP apparatus, 0.01N HCl, 900 ml, and 100 rpm.

FIG. 1(b) shows a release profile of controlled release dosage forms of zolpidem of example 1, in Type II USP apparatus 0.01N HCl, 900 ml, and 50 rpm.

FIG. 2(a) shows a release profile of controlled release dosage forms of zolpidem of example 2, in Type I USP apparatus, 0.01N HCl, 900 ml, and 100 rpm.

FIG. 2(b) shows a release profile of controlled release dosage forms of zolpidem of example 2, in Type II USP apparatus 0.01N HCl, 900 ml, and 50 rpm.

FIG. 3(a) shows a release profile of controlled release dosage forms of zolpidem of example 3, in Type I USP apparatus, 0.01N HCl, 900 ml, and 100 rpm.

FIG. 3(b) shows a release profile of controlled release dosage forms of zolpidem of example 3, in Type II USP apparatus 0.01N HCl, 900 ml, and 50 rpm.

FIG. 4 (a) shows a release profile of controlled release dosage forms of zolpidem of example 4, in Type I USP apparatus, 0.01N HCl, 900 ml, and 100 rpm.

FIG. 4 (b) shows a release profile of controlled release dosage forms of zolpidem of example 3, in Type II USP apparatus 0.01N HCl, 900 ml, and 50 rpm.

### DETAILED DESCRIPTION OF THE INVENTION

The dosage forms according to the invention typically contain from 4 to 16 mg of zolpidem as zolpidem base, and preferably 6 to 12 mg of zolpidem as zolpidem base. The zolpidem may be incorporated as the base, or as a pharmaceutically acceptable salt of zolpidem. Among dosage forms comprising a salt of zolpidem rather than the zolpidem base, according to the invention, those comprising zolpidem hemitartrate are especially preferred.

These pharmaceutically acceptable organic acid can be chosen for example among maleic, tartaric, malic, fumaric, lactic, citric, adipic or succinic acid and their acid salts where these exist, in the form of racemates or isomers, where these exist. According to the invention, acids particularly preferred are tartaric, fumaric, citric, and succinic and their acid salts.

The formulation of the present invention is not restricted to any particular type of formulation. Thus, various types of controlled or sustained release type formulations may be used for embodying the present invention, such as, for example, osmotic tablets, gel matrix tablets, coated beads, etc.

Various formulations, not limiting the scope of the present invention, illustrating the invention are described hereafter:

The matrix materials useful for this embodiment are generally water-insoluble materials such as waxes, cellulose, or other water-insoluble polymers. If needed, the matrix materials may optionally be formulated with water-soluble materials, which can be used as binders or as permeability-modifying agents. Matrix materials useful for the manufacture of these dosage forms include microcrystalline cellulose such as Avicel (registered trademark of FMC Corp., Philadelphia, Pa.), including grades of microcrystalline cellulose to which binders such as hydroxypropyl methyl cellulose have been added, waxes such as paraffin, modified vegetable oils, carnauba wax, hydrogenated castor oil, beeswax, and the like, as well as synthetic polymers such as poly(vinyl chloride), poly(vinyl acetate), copolymers of vinyl acetate and ethylene, polystyrene, and the like. Water-soluble binders or release modifying agents, which can optionally be formulated into the matrix, include water-soluble polymers such as hydroxypropyl cellulose (HPC), hydroxypropyl methyl cellulose (HPMC), methylcellulose, poly (N-vinyl-2-pyrrolidinone) (PVP), poly (ethylene oxide) (PEO), poly (vinyl alcohol) (PVA), xanthan gum, carrageenan, and other such natural and synthetic materials. In addition, materials which function as release-modifying agents include water-soluble materials such as sugars or salts. Preferred water-soluble materials include lactose, sucrose, glucose, and mannitol, as well as HPC, HPMC, and PVP.

Another common type of controlled release formulation that may be used for the purposes of the present invention comprises an inert core, such as a sugar sphere, coated with an inner drug-containing layer and an outer membrane layer controlling drug release from the inner layer. A “sealcoat” may be provided between the inert core and the layer containing the active ingredient. When the core is of a water-soluble or water-swellable inert material, the sealcoat is preferably in the form of a relatively thick layer of a water-insoluble polymer. Such a controlled release bead may thus comprise: (i) a core unit of a substantially water-soluble or water-swellable inert material; (ii) a first layer on the core unit of a substantially water-insoluble polymer; (iii) a second layer covering the first layer and containing an active ingredient; and (iv) a third layer on the second layer of polymer effective for controlled release of the active ingredient, wherein the first layer is adapted to control water penetration into the core. The number of layers can be manipulated in such a way so that the current object of the invention is achieved.

The controlled release beads may be provided in a multiple unit formulation, such as a capsule or a tablet. The cores are preferably of a water-soluble or swellable material, and may be any such material that is conventionally used as cores or any other pharmaceutically acceptable water-soluble or water-swellable material made into beads or pellets. The cores may be spheres of materials such as sucrose/starch (Sugar Spheres NF), sucrose crystals, or extruded and dried spheres typically comprised of excipients such as microcrystalline cellulose and lactose.

The layer containing the active ingredient may be comprised of the active ingredient (drug) with or without a polymer as a binder. The binder, when used, is usually hydrophilic but may be water-soluble or water-insoluble. Exemplary polymers to be used in the layer containing the active drug are hydrophilic polymers such as polyvinylpyrrolidone (PVP), polyalkylene glycol such as polyethylene glycol, gelatin, polyvinyl alcohol, starch and derivatives thereof, cellulose derivatives, such as hydroxypropylmethyl cellulose (HPMC), hydroxypropyl cellulose, carboxymethyl cellulose, methyl cellulose, ethyl cellulose, hydroxyethyl cellulose, carboxyethyl cellulose, carboxymethylhydroxyethyl cellulose, acrylic acid polymers, polymethacrylates, or any other pharmaceutically acceptable polymer.

Suitable polymers for use for controlling the drug release may be selected from water-insoluble polymers or polymers with pH-dependent solubility, such as, for example, ethyl cellulose, hydroxypropylmethyl cellulose phthalate, cellulose acetate phthalate, cellulose acetate trimellitate, polymethacrylates, or mixtures thereof, optionally combined with plasticizers, such as those mentioned above. Optionally, the controlled release layer comprises, in addition to the polymers above, another substance(s) with different solubility characteristics, to adjust the permeability, and thereby the release rate, of the controlled release layer. Exemplary polymers that may be used as a modifier together with, for example, ethyl cellulose include: HPMC, hydroxyethyl cellulose, hydroxypropyl cellulose, methylcellulose, carboxymethylcellulose, polyethylene glycol, polyvinylpyrrolidone (PVP), polyvinyl alcohol, polymers with pH-dependent solubility, such as cellulose acetate phthalate or ammonio methacrylate copolymer and methacrylic acid copolymer, or mixtures thereof. Additives such as sucrose, lactose and pharmaceutical grade surfactants may also be included in the controlled release layer, if desired.

The above controlled release beads and formulation, respectively may be produced by a method comprising the following steps: a) providing a core unit of a substantially water-soluble or water-swellable material; b) applying a first layer of a substantially water-insoluble polymer to said core; c) applying onto said first layer, a second layer comprising an active ingredient and optionally a polymer binder; and d) applying onto said second layer, a third polymer layer effective for controlled release of the active ingredient; wherein the amount of material in said first layer is selected to provide a layer thickness that permits control of water penetration into the core.

The diluents to be used in the above formulation include but are not limited to starch cellulose, calcium sulphate, calcium carbonate, dicalcium phosphate, lactose, dextrose, sucrose, dextrates, mannitol, maltodextrin, methylcellulose, and polyethylene glycol.

A third class of zolpidem controlled-release dosage forms includes the osmotic delivery devices or “osmotic pumps” as they are known in the art. Osmotic pumps comprise a core containing an osmotically effective composition surrounded by a semi permeable membrane. The term “semi permeable” in this context means that water can pass through the membrane, but solutes dissolved in water cannot. In use, when placed in an aqueous environment, the device imbibes water due to the osmotic activity of the core composition. Owing to the semi permeable nature of the surrounding membrane, the contents of the device (including the drug and any excipients) cannot pass through the non-porous regions of the membrane and are driven by osmotic pressure to leave the device through an opening or passageway pre-manufactured into the dosage form or, alternatively, formed in situ in the GI tract as by the bursting of intentionally-incorporated weak points in the coating under the influence of osmotic pressure. The osmotically effective composition includes water-soluble species, which generate a colloidal osmotic pressure, and water-swellable polymers. The drug itself (if highly water-soluble) may be an osmotically effective component of the mixture.

Materials useful for forming the semi permeable membrane include polyamides, polyesters, and cellulose derivatives. Preferred are cellulose ethers and esters. Especially preferred are cellulose acetate, cellulose acetate butyrate, and ethyl cellulose. Especially useful materials include those which spontaneously form one or more exit passageways, either during manufacturing or when placed in an environment of use. These preferred materials comprise porous polymers, the pores of which are formed by phase inversion during manufacturing, as described above, or by dissolution of a water-soluble component present in the membrane.

A preferred embodiment of this class of osmotic delivery devices consists of a coated bi-layer tablet. The coating of such a tablet comprises a membrane permeable to water but substantially impermeable to zolpidem and excipients contained within. The coating contains one or more exit passageways in communication with the zolpidem-containing layer for delivering the drug composition. The tablet core consists of two layers: one layer containing the zolpidem composition and another layer consisting of an expandable hydrogel, with or without additional osmotic agents. When placed in an aqueous medium, the tablet imbibes water through the membrane, causing the zolpidem composition to form a dispensible aqueous composition, and causing the hydrogel layer to expand and push against the zolpidem composition, forcing the zolpidem composition out of the exit passageway.

The rate of zolpidem delivery is controlled by such factors as the permeability and thickness of the coating, the water activity of the hydrogel layer, and the surface area of the device. Those skilled in the art will appreciate that increasing the thickness of the coating will reduce the release rate, whereas increasing the permeability of the coating or the water activity of the hydrogel layer or the surface area of the device will increase the release rate.

Exemplary materials, which are useful to form the zolpidem composition, in addition to the zolpidem itself, include hydroxypropyl methylcellulose, poly (ethylene oxide), poly (N-vinyl-2-pyrrolidinone) or PVP, and other pharmaceutically acceptable carriers. In addition, osmagens such as sugars or salts, especially sucrose, mannitol, or sodium chloride, may be added. Materials, which are useful for forming the hydrogel layer, include sodium carboxymethyl cellulose, poly (ethylene oxide), poly (acrylic acid), sodium (poly-acrylate) and other high molecular-weight hydrophilic materials. Particularly useful are poly (ethylene oxide) having a molecular weight from about 4,000,000 to about 7,500,000 and sodium carboxymethyl cellulose having a molecular weight of about 200,000 to about 1,000,000. Materials which are useful for forming the coating are cellulose esters, cellulose ethers, and cellulose ester-ethers. Preferred are cellulose acetate and ethylcellulose. The exit passageway must be located on the side of the tablet containing the zolpidem composition. There may be more than one such exit passageway. The exit passageway may be produced by mechanical or by laser drilling, or by creating a difficult-to-coat region on the tablet by use of special tooling during tablet compression. The rate of zolpidem delivery from the device may be optimized so as to provide a method of delivering zolpidem to a mammal for optimum therapeutic effect.

The solid oral dosage form can be prepared by techniques well known in the art and contains a therapeutically useful amount of zolpidem plus such excipients as are necessary to form the tablet by such techniques.

The solid oral dosage forms of the present invention may be prepared by conventional techniques for example wet granulation, compaction or direct compression.

### Example 1

| S. No | Ingredient | (%) w/w |
|---|---|---|
| 1 | Zolpidem Tartrate | 5% |
| 2 | Lactose Monohydrate | 59% |
| | (Pharmatose DCL 11) | |
| 3 | Microcrystalline Cellulose | 20% |
| | (Avicel PH102) | |
| 4 | Hypromellose | 13% |
| | (Methocel K100LVCR) | |
| 5 | Hypromellose | — |
| | (Methocel E5) | |
| 6 | Colloidal Silicondioxide | 2% |
| | (Aerosil 200) | |
| 7 | Magnesium Stearate | 1% |
| 8 | Film Coating | 3% |
| | (Opadry) | |

### Brief Manufacturing Process

1. Sifting: Sift 1, 3 & 4 together through sifted through a suitable seive (ASTM). Sift this dry mix and some qty of Lactose together through sifted through a suitable seive.
2. Dry mixing: Blend the sifted material of the above step in suitable blender for required time to get uniform dry blend.
3. Blending & Lubrication: Add to the above dry blend, Aerosil (previously sifted through a suitable seive and Magnesium stearate (previously sifted through a suitable seive) and blend for required time.
4. Compression: Compress tablet using suitable punch at sufficient hardness.
5. Coating: Coat the tablets using aqueous dispersion of coating material.

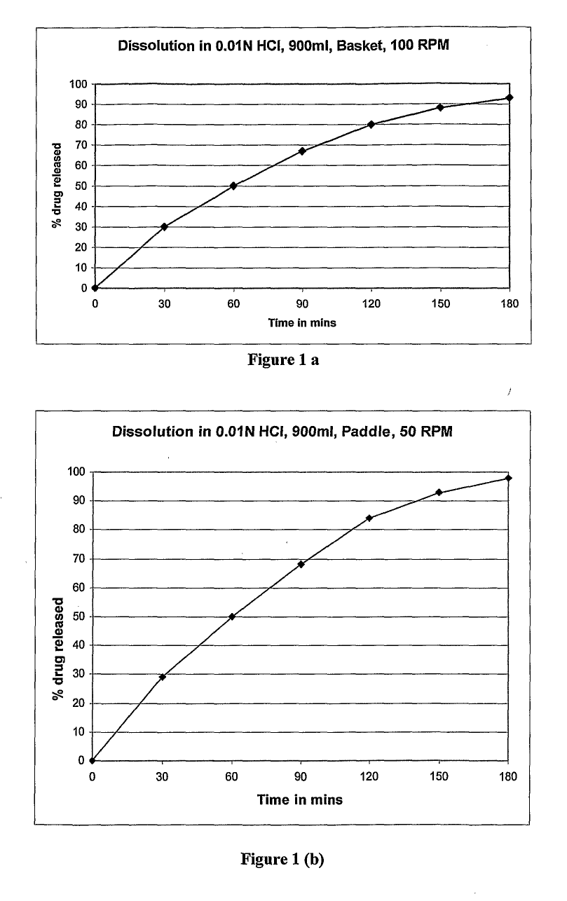

The in vitro dissolution profiles of the tablets were established using the Apparatus I and II of the United States Pharmacopoeia. The dissolution media employed was 900 ml hydrochloric acid 0.01 M, maintained at 37.+−.0.5.degree. C. Stirring was by the basket method (100 rpm) (FIG. 1a) and paddle (50 rpm) (FIG. 1b) and the percentage dissolved was determined by measurement of the UV absorbance at 270 nm. The results are shown in FIG. 1.

### Example 2

### Preparation of Controlled Release Beads and Capsules of Zolpidem

| S. No | Ingredient | (%) w/w |
|---|---|---|
| 1 | Zolpidem Tartrate | 5% |
| 2 | Sugar Spheres, 20-25 # | 70% |
| 3 | Surelease ® | 20% |
| 4 | Hypromellose 5Cps | 5% |

Optionally, a fourth layer may be applied to the bead before drying by Wurster coating. Fourth layer: HPMC; comprises about 1% w/w of the final bead; purpose: decrease tackiness of beads for subsequent processing (curing and capsule filling).

### Brief Manufacturing Process

Core: Starch-containing sugar sphere (commercially available); comprises 70% w/w of the final bead; purpose: coating substrate;

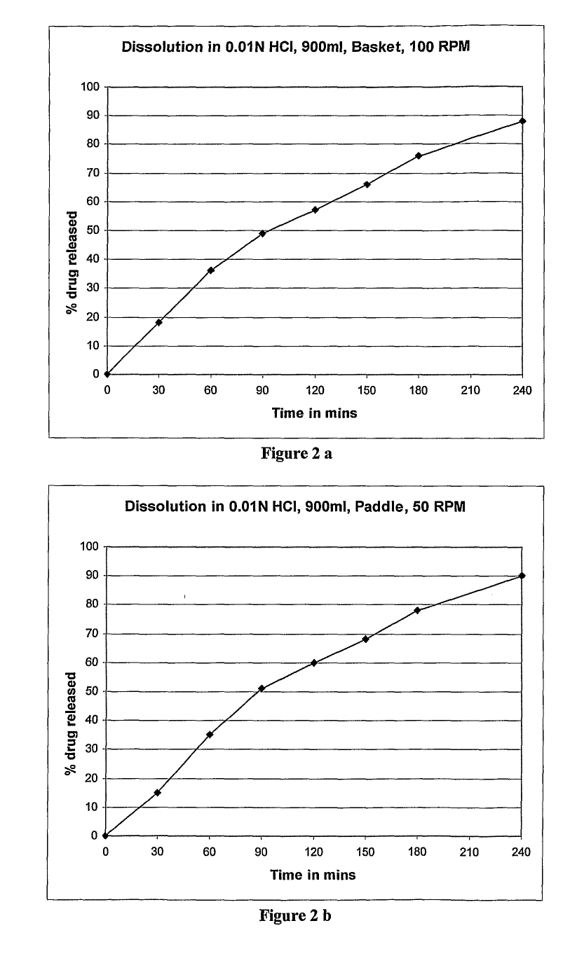

First Surelease® “Sealcoat” (Surelease® is an aqueous film-coating layer: dispersion, about 25% solids, consisting primarily of ethylcellulose plasticized with fractionated coconut oil, and manufactured by Colorcon, Inc, USA); comprises about 12% w/w of the final bead; purpose: to provide more consistent core surface; during drug release phase maximize time that drug is saturated inside bead and minimize osmotic effects; control drug release rate together with the third layer;
Second Zolpidem hemitartrate/hydroxypropylmethylcellulose (HPMC); layer: comprises about 3% w/w of the final bead; ratio of Zolpidem:HPMC is 4:1; purpose: drug supply;
Third Surelease®/HPMC; comprises about 12% w/w of the final layer: bead; ratio of Surelease®:HPMC is 6:1; purpose: drug release rate control; Beads with a three-layer coating having the above characteristics were prepared as follows:
1200 g of sugar spheres, 20-25 mesh, were charged into a Wurster fluid bed and sequentially coated at a nominal product temperature of 36 to 40.degree. C. with the following three coating liquids: (1) a Surelease® Seal coating liquid prepared by mixing 140 g of Surelease® with 1000 g of isopropyl alcohol; (2) a drug-containing solution prepared by first dissolving 85.5 g of Zolpidem hemitartrate in 2000 g of purified water, and then mixing the solution with 15.0 g of hydroxypropylmethyl cellulose (HPMC) 5 cP; and (3) a sustained release coating liquid prepared by mixing 70 g of HPMC 5 cP with 375 g of purified water, and then mixing with 100 g of Surelease®. After tray drying for a suitable period of time at 60.degree. C., the coated spheres were filled into size #4 or size #3 hard gelatin capsules to obtain 12.5 mg and 6.25 mg of Zolpidem hemitartrate capsules, respectively, of the composition: The in vitro dissolution profiles of the tablets were established using the Apparatus I and II of the United States Pharmacopoeia. The dissolution media employed was 900 ml hydrochloric acid 0.01 M, maintained at 37.+−.0.5.degree. C. Stirring was by the paddle method (50 rpm) and basket (100 rpm). The percentage dissolved was determined by measurement of the UV absorbance at 270 nm. The results are shown in FIG. 2.

### Example 3

| S. No | Ingredient | (%) w/w |
|---|---|---|
| 1 | Zolpidem Tartrate | 5% |
| 2 | Lactose | 45% |
| 3 | Microcrystalline cellulose | 15% |
| 4 | Magnesium stearate | 1% |
| 5 | Polyethylene oxide | 10% |
| 6 | Lactose | 16% |
| 7 | Polyvinylpyrrolidine | 5% |
| 8 | Colloidal Silicon dioxide | 2% |
| 9 | Magnesium stearate | 1% |

### Brief Manufacturing Process

### Part a)

1 Sift 1, 2 & 3 together through a suitable sieve.
2 Blend the sifted material of the above step in suitable blender for required time to get uniform dry blend.
3 Add to the above dry blend, and Magnesium stearate (previously sifted through a suitable sieve) and blend for required time.

### Part b)

4 Sift 5, 6 & 7 together through sifted through a suitable seive
5 Blend the sifted material of the above step 4 in suitable blender for required time to get uniform dry blend.
6. Add to the above dry blend, Aerosil (previously sifted through a suitable seive and Magnesium stearate (previously sifted through a suitable seive) and blend for required time.
7. Compress part a) and part b) to form Bilayer tablets.

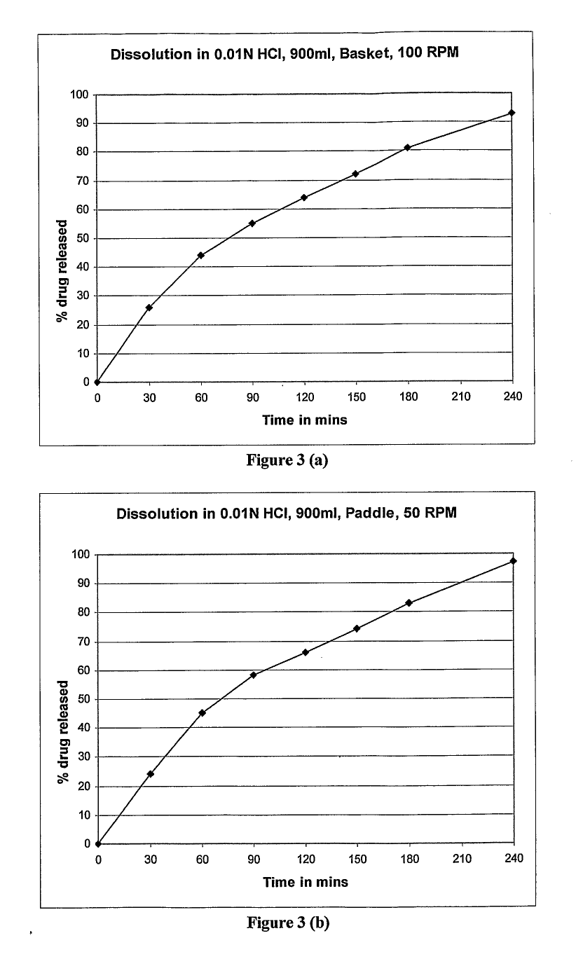

The in vitro dissolution profiles of the tablets were established using the Apparatus I and II of the United States Pharmacopoeia. The dissolution media employed was 900 ml hydrochloric acid 0.01 M, maintained at 37.+−.0.5.degree. C. Stirring was by the paddle method (50 rpm) and basket (100 rpm). The percentage dissolved was determined by measurement of the UV absorbance at 270 nm. The results are shown in FIG. 3.

### Example 4

| S. No | Ingredient | (%) w/w |
|---|---|---|
| 1 | Zolpidem Tartrate | 5% |
| 2 | Lactose | 30% |
| 3. | Di Calcium Phosphate | 20% |
| 4 | Microcrystalline cellulose | 10% |
| 5 | Magnesium stearate | 1% |
| 6 | Polyethylene oxide | 10% |
| 7 | Lactose | 16% |
| 8 | Polyvinylpyrrolidine | 5% |
| 9 | Colloidal Silicon dioxide | 2% |
| 10 | Magnesium stearate | 1% |

### Brief Manufacturing Process

### Part a)

1 Sift 1, 2, 3 and 4 together through a suitable sieve.
2 Blend the sifted material of the above step in suitable blender for required time to get uniform dry blend.
3 Add to the above dry blend, and Magnesium stearate (previously sifted through a suitable sieve) and blend for required time.

### Part b)

4 Sift 6, 7 and 8 together through a suitable seive
5 Blend the sifted material of the above step 4 in suitable blender for required time to get uniform dry blend.
6 Add to the above dry blend, Aerosil (previously sifted through a suitable seive and Magnesium stearate (previously sifted through a suitable seive) and blend for required time.
7 Compress part a) and part b) to form Bilayer tablets.

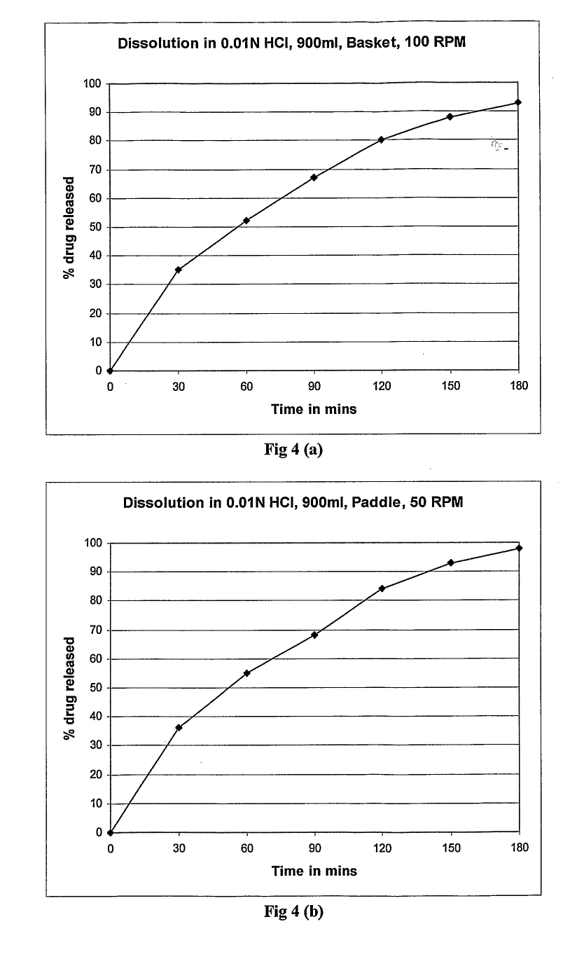

The in vitro dissolution profiles of the tablets were established using the Apparatus I and II of the United States Pharmacopoeia. The dissolution media employed was 900 ml hydrochloric acid 0.01 M, maintained at 37.+−.0.5.degree. C. Stirring was by the paddle method (50 rpm) and basket (100 rpm). The percentage dissolved was determined by measurement of the UV absorbance at 270 nm. The results are shown in FIG. 4

1. A pharmaceutical controlled-release dosage form of zolpidem or a salt thereof having a dissolution profile when measured in a type I or II dissolution apparatus according to the U.S. Pharmacopoeia in 0.01M hydrochloric acid buffer at 37.degree. C., such that less than 40% is released at the end of 30 minutes.

2. A pharmaceutical controlled-release dosage form according to claim 1 containing a pharmaceutically acceptable organic acid selected from tartaric, malic, fumaric, lactic, citric, adipic and succinic acids or their acid salts.

3. A pharmaceutical controlled-release dosage form according to claim 1 wherein zolpidem is present as zolpidem hemitartrate.

4. A pharmaceutical composition according to claim 1 in a dosage form selected from capsules, tablets, multilayer tablets, osmotic tablets, gel matrix, coated beads and multicoated tablets.

5. A pharmaceutical composition according to claim 4 in the form of a capsule comprising controlled release pellets.

6. A pharmaceutical composition according to claim 4 in the form of a tablet comprising the drug imbedded in a matrix wherein the matrix comprises of hydrophobic polymer and/or hydrophilic polymer and optionally a release modifying agent.

7. A pharmaceutical composition according to claim 4 in the form of a multilayer tablet comprising: (i) one or more controlled release layers, comprising the drug and a hydrophilic polymer and/or hydrophobic polymers.

8. A pharmaceutical composition according to claim 4 in the form of a osmotic tablet comprising: (i) a core containing an osmotically effective composition and drug; (ii) and a semi-permeable membrane.

9. A pharmaceutical composition according to claim 4 in the form of a osmotic tablet comprising: (i) a core containing an osmotically effective composition; (ii) and an expandable hydrogel polymer.

10. A pharmaceutical composition according to claim 4 wherein the coated beads is comprising (i) an inert core; (ii) a drug containing second layer and (iii) a release controlling membrane and optionally (iv) a seal coat.

11. A pharmaceutical composition according to claim 4 in the form of a multicoated tablet comprising: (i) a core comprising the drug, (ii) a polymer coating layer giving controlled release of the drug from this core.

12. A pharmaceutical controlled-release dosage form of Zolpidem or a salt thereof having a dissolution profile characterized such that 90% of the total amount of drug is released between 2.5 and 7.5 hours and preferably between 3.0 to 4.5 hours.

13. A pharmaceutical controlled-release dosage form of Zolpidem or a salt thereof having Cmax in the range from 65 ng/ml to about 230 ng/ml.

14. A pharmaceutical controlled-release dosage form of Zolpidem or a salt thereof having mean AUC₀₋ₜ in the range from 255 ng*hr/ml to about 1610 ng*hr/ml

15. A pharmaceutical controlled-release dosage form according to claim 1 wherein the in vitro profile of dissolution is measured in a type I or II dissolution apparatus according to the U.S. Pharmacopoeia in 0.01M hydrochloric acid buffer at 37.degree. C. stirred at a rate of about 50-100 rpm.
